(19) United States
(12) Patent Application Publication
(10) Pub. No.: US 2016/0060324 A1
(43) Pub. Date: Mar. 3, 2016

(54) Methods and Compositions for Treating Bleeding Disorders

(71) Applicants: James C. PAULSON, Del Mar, CA (US); Matthew MACAULEY, San Diego, CA (US); Fabian PFRENGLE, Berlin (DE); THE SCRIPPS RESEARCH INSTITUTE, La Jolla, CA (US)

(72) Inventors: James C. Paulson, Del Mar, CA (US); Matthew Macauley, San Diego, CA (US); Fabian Pfrengle, Berlin (DE)

(21) Appl. No.: 14/786,419

(22) PCT Filed: Apr. 18, 2014
(86) PCT No.: PCT/US2014/034623

§ 371 Date: Oct. 22, 2015

Related U.S. Application Data
(60) Provisional application No. 61/814,526, filed on Apr. 22, 2013.

(51) Int. Cl. C07K 14/755; A61K 38/37; A61K 47/48

(52) U.S. Cl. CPC C07K 14/755; A61K 47/48276; A61K 38/37

(57) ABSTRACT

The present invention provides immune conjugates for inducing antigen specific immune tolerance to coagulation Factor VIII. The immune conjugates contain a FVIII protein or antigenic fragment that is conjugated to a binding moiety for a sialic acid binding Ig-like lectin (Siglec) expressed on B cells. The invention also provides methods of using the FVIII immune conjugates to induce immune tolerance to FVIII in a subject. Additionally provided in the invention are methods for treating bleeding disorders such as hemophilia A via the use of the FVIII immune conjugates and an unconjugated FVIII with coagulating activity.

### CROSS-REFERENCE TO RELATED APPLICATIONS

The subject patent application claims the benefit of priority to U.S. Provisional Patent Application No. 61/814,526, filed Apr. 22, 2013. The full disclosure of the priority application is incorporated herein by reference in its entirety and for all purposes.

### STATEMENT OF GOVERNMENT SUPPORT

This invention was made with U.S. government support under Grant Nos. R01AI050143 and R01AI099141 awarded by the National Institutes of Health. The government has certain rights in this invention.

### BACKGROUND OF THE INVENTION

Unwanted humoral immune responses to protein antigens are responsible for numerous medical conditions in the areas of autoimmunity, transplantation, allergies, and biotherapeutics. Current treatment options largely rely on immunosuppressive drugs or immunodepletion therapy, but these approaches can compromise immunity. A more desirable approach is to silence or delete the antigen-reactive lymphocytes in a manner that preserves protective immunity. Several approaches for inducing antigen-specific tolerance have shown some promise. One, termed antigen-specific immunotherapy (SIT), involves sustained high dose of the antigen administered over the course of months to years. Another involves the expression or attachment of the antigen to syngeneic cells. In all these approaches, the mechanism of tolerance induction is thought to be a direct effect on antigen-specific T cells or an induction of regulatory T cells.

As an alternative to T cell directed therapy, targeting the antigen-reactive B-cells offers a more direct approach for systematic induction of humoral tolerance to the desired antigens. Indeed, B-cells are the progenitors of antibody-secreting plasma cells and participate in non-humoral immune responses through the release of cytokines. However, methods to directly tolerize B-cells in an antigen-specific manner are lacking. For example, the development of inhibitors is the most serious complication in patients with hemophilia with a high risk of mortality from fatal bleeding. Currently, the only option to achieve immune tolerance in patients with hemophilia A (congenital FVIII-deficiency) and inhibitors is immune tolerance induction (ITI), where high doses of FVIII are administered for prolonged periods of time. Treatment can take 2 years, remains unsuccessful in approximately 30% of patients, is extraordinarily costly, and cannot be used in a prophylactic manner to suppress the initial development of inhibitory antibodies.

Thus, there is a need in the art for safer and more effective means for inducing immune tolerance in treating or preventing bleeding disorders such as hemophilia A. The instant invention addresses this and other needs.

### SUMMARY OF THE INVENTION

In one aspect, the invention provides compounds or immune conjugates for inducing antigen specific immune tolerance to coagulating Factor III (FVIII) protein. The compounds typically contain a FVIII protein or antigenic fragment thereof that is conjugated to a binding moiety for a sialic acid binding Ig-like lectin (Siglec). In some compounds, the FVIII or antigenic fragment thereof is conjugated to the binding moiety via a liposome. In some other compounds, the FVIII antigen is covalently conjugated to the binding moiety, e.g., via a linker moiety. Some preferred compounds contain a human FVIII protein or antigen. In some preferred embodiments, the binding moiety in the immune conjugates is a ligand for a Siglec expressed on B lymphocytes, e.g., CD22 or Siglec-G/10. In some embodiments, the binding moiety contains a glycan ligand for the Siglec. Some specific examples of binding moieties that can be used in the immune conjugates include 9-N-biphenylcarboxyl-NeuAcα2-6Galβ1-4GlcNAc (6′-BPCNeuAc), NeuAcα2-6Galβ1-4GlcNAc, and NeuAcα2-6Galβ1-4(6-sulfo)GlcNAc.

In a related aspect, the invention provides methods for inducing immune tolerance to Factor VIII (FVIII) in a subject. These methods entail administering to the subject a therapeutically effective amount of a compound that contains a Factor VIII protein or antigenic fragment thereof that is conjugated to a binding moiety for a sialic acid binding Ig-like lectin (Siglec) expressed on B lymphocytes. In some methods, the FVIII antigen in the administered compound is conjugated to the binding moiety via a liposome. In some other methods, the FVIII antigen in the administered compound is covalently conjugated to the binding moiety via a linker. Some preferred methods are directed to targeting the FVIII antigen to CD22 or Siglec-G/10 on B cells. In some methods, the binding moiety in the administered immune conjugates contains a glycan ligand for the Siglec. Examples of such binding moiety include 9-N-biphenylcarboxyl-NeuAcα2-6Galβ1-4GlcNAc (6′-BPCNeuAc), NeuAcα2-6Galβ1-4GlcNAc, and NeuAcα2-6Galβ1-4(6-sulfo)GlcNAc. Some preferred methods are directed to tolerize a human subject. In such methods, the FVIII antigen present in the administered immune conjugates is a human FVIII protein or antigenic fragment. In some methods, the administered compounds contain human FVIII that is conjugated to 9-N-biphenylcarboxyl-NeuAcα2-6Galβ1-4GlcNAc (6′-BPCNeuAc) via a liposome. Some of the methods for inducing immune tolerance to FVIII are specifically intended for subjects afflicted with a bleeding disorder such as hemophilia A. Typically, the FVIII immune conjugate or compound are administered to a subject in a pharmaceutical composition.

In another related aspect, the invention provides methods for treating hemophilia A in a subject. These methods involve administering to a subject in need of treatment a FVIII immune conjugate in conjunction with an unconjugated FVIII protein or variant with coagulation activity. The administered FVIII immune conjugate typically contains a FVIII protein or antigenic fragment that is conjugated to a glycan ligand for a B lymphocyte sialic acid binding Ig-like lectin (Siglec). In some embodiments, the FVIII immune conjugate is administered to the subject prior to administration of the unconjugated FVIII protein or variant. In some embodiments, the FVIII protein or antigen in the conjugate compound is conjugated to the glycan ligand via a liposome. In some other embodiments, the FVIII protein in the administered conjugate compound is covalently conjugated to the glycan ligand via a linker. Some preferred methods are directed to treating a human subject. In these methods, the FVIII protein in the administered conjugate compound is preferably human FVIII. In various embodiments, the co-administered unconjugated FVIII can be either recombinant or plasma derived human FVIII. In some preferred embodiments, the administered FVIII immune conjugate targets CD22 or Siglec-10 on B cells in the human subject. In these embodiments, the glycan ligands used in the immune conjugates can be, e.g., 9-N-biphenylcarboxyl-NeuAcα2-6Galβ1-4GlcNAc (6′-BPCNeuAc), NeuAcα2-6Galβ1-4GlcNAc, or NeuAcα2-6Galβ1-4(6-sulfo)GlcNAc.

A further understanding of the nature and advantages of the present invention may be realized by reference to the remaining portions of the specification and claims.

### DESCRIPTION OF THE DRAWINGS

FIGS. 1A-1F show induction of tolerance with liposomes displaying antigen and CD22 ligands. (A) Schematic of STALs; Siglec-engaging Tolerance-inducing Antigenic Liposomes. (B) Chemical structures of CD22 ligands used for studies in mice. (C,D) CD22-dependent induction of tolerance to a T-independent (NP; C) and a T-dependent antigen (HEL; D). WT or CD22KO mice were treated on day 0 (open arrow) as shown and challenged with the immunogenic liposomes on days 15 and 30 (closed arrow). Data represents mean+/−s.e.m. (n=8-10). (E) Titration of ^{BPA}NeuGc and NeuGc on STALs. Titers were determined after two challenges with immunogenic liposomes on days 15 and 30 (n=4). (F) Mice were tolerized to HEL at different times relative to the challenge and titers were determined two weeks after challenge with immunogenic liposomes and are plotted as percentage relative to immunization of naïve mice (n=4). Data represents mean+/−s.e.m. (n=4).

FIGS. 2A-2F show that STALs strongly inhibit BCR signaling and cause apoptosis. (A) Calcium flux in IgM^{HEL} B-cells stimulated with the indicated liposomes. (B) CD86 upregulation of IgM^{HEL} B-cells 24 hr after stimulation with the indicated liposomes. (C) In vitro proliferation of CTV-labeled IgM^{HEL} B-cells three days after simulation with the indicated liposomes. (D) AnnexinV versus PI staining of IgM^{HEL} B-cells treated for 24 hr with the indicated liposomes. For quantification over time, the %PI⁻AnnexinV⁻ (live) cells are expressed relative to the controls treated with naked liposomes normalized to 100% at each time point and plotted as the mean+/−s.e.m. (n=3). (E) In vivo proliferation of adoptively-transferred CFSE-labeled IgM^{HEL} B-cells four days after immunization with the indicated liposomes. The same number of total splenocytes were analyzed for each condition (1×10⁶) and gated through the IgM^{a+}Ly5^{a+} population. (F) Analysis of the number of adoptively-transferred Ly5^{a+}IgM^{HEL} B-cells remaining in the spleen of recipient mice 12 days after immunization with the indicated liposomes. Quantitation represents mean+/−s.e.m (n=4).

FIGS. 3A-3B show that a CD22-dependent tolerogenic program inhibits basal signaling in the Akt survival pathway and drives nuclear import of FoxO1. (A) Western blot analysis of BCR signaling components in WT and CD22KO IgM^{HEL} B-cells 30 minutes after stimulation of cells with the indicated liposomes or PBS as a control. STALs inhibit phosphorylation of signaling components of all major BCR signaling pathways and induce hypo-phosphorylation of Akt and FoxO1 in WT B-cells, but not CD22 deficient Igm^{HEL} B-cells. Data is a subset of Figure S4. (B) Analysis of FoxO1 staining in IgM^{HEL} B-cells by confocal microscopy. Cells were stimulated for 2 hr stained with the indicated liposomes and stained with anti-FoxO1, phalloidin, and DAPI. Inserts are a representative cell at three-times the magnification.

FIGS. 4A-4D show antigen-specific tolerization of mice to strong T-dependent antigens. (A) Tolerization of OVA in C57BL/6J mice. (B) Tolerization of MOG (residues 1-120) in Balb/c mice. (C) Tolerization of FVIII in Balb/c. (D) Tolerization is antigen-specific. Balb/c mice tolerized to HEL or OVA have normal responses to other antigen. Mice were immunized on day 0 with the indicated conditions, challenged on day 15 with immunogenic liposomes, and titers (IgG₁) determined two weeks later on day 29. All data represents mean+/−s.e.m. (n=4).

FIGS. 5A-5B show that immune tolerization to FVIII prevents bleeding in FVIII-deficient mice. (A) WT or FVIII-deficient mice were dosed on day 0 and 15 with immunogenic liposomes (immunogen), STALs, or left untreated. On day 30, mice were reconstituted with recombinant human FVIII (rhFVIII) at 50 U/kg or saline. FVIII-deficient mice treated with STALs had significantly less blood loss (μL/g) over 20 minutes following tail clip than mice initially treated with immunogenic liposomes. Percent bleeding protection (dashed line) represents blood loss <9.9 μl/g as defined by mean plus 3 SDs in WT Balb/c mice. (B) FVIII-titers in the three reconstituted groups demonstrate that bleeding prevention is accompanied by a significant reduction in anti-FVIII antibodies. Data represents mean+/−s.e.m. A two-tailed Student's t-test was used to establish the level of significance; no statistical difference (n.s.) is defined by a P value greater than 0.05.

FIGS. 6A-6F show that STALS induce apoptosis in naive and memory human B-cells. (A) Structure of the high affinity human CD22 ligand ^{BPC}NeuAc. (B-D) Activation of naive and memory human B-cells is inhibited by co-presentation of ^{BPC}NeuAc with cognate antigen (anti-IgM or anti-IgG, respectively) on liposomes, as judged by calcium flux (B), Western blot analysis of BCR signaling components (C), and CD86 upregulation (D). (E) Liposomes displaying cognate antigen and hCD22 ligands decrease viability of both naive and memory human B-cells. Data represents mean+/−s.e.m (n=3). A two-tailed Student's t-test was used to establish the level of significance. (F) Staining of naive (red) and memory (blue) human B-cells with anti-CD22 or isotype control (grey) antibodies. Data is representative from three healthy donors.

### DETAILED DESCRIPTION

### I. Overview

The present invention is predicated in part on the present inventors' discovery that physically linking CD22 with B-cell receptor (BCR) can induce tolerance to a specific protein antigen, Factor VIII (FVIII), in a hemophilia mouse model. As detailed herein, the inventors observed that enforced association of CD22 with BCR, e.g., via Siglec-engaging tolerance-inducing antigenic liposomes (STALs), prevented formation of inhibitory FVIII antibodies. This allowed for effective administration of FVIII to hemophilia mice to prevent bleeding.

As detailed herein, the inventors exploited the natural mechanisms that suppress B-cell activation. B-cells express a host of B-cell receptor (BCR) inhibitory co-receptors, which help set a threshold for activation. Among them are CD22 and Siglec-G (Siglec-10 in humans), members of the Siglec (sialic acid binding Ig-like lectins) immunoglobulin family that recognize sialic acid-containing glycans of glycoproteins and glycolipids as ligands. To enforce ligation of the BCR and CD22 for inducing tolerance to protein antigens, the inventors employed immune conjugates containing a FVIII protein and a binding agent for CD22, e.g., a liposomal nanoparticle that displays both the protein antigen and the CD22 ligand. It was found that these Siglec-engaging tolerance-inducing antigenic liposomes (STALs) induce antigen-specific tolerance to T-dependent antigens in mice via deletion of the antigen-reactive B-cells by apoptosis. The utility of this platform for preventing an undesired antibody response is illustrated by complete suppression of anti-FVIII antibodies in a hemophilia mouse model following challenge with human FVIII (hFVIII). In addition, induced tolerance to FVIII and suppression of anti-FVIII antibodies enabled protection of mice from bleeding in a tail cut assay following administration of hFVIII. Further, STALs also induced a tolerogenic program in human primary B-cells within both the naïve and memory compartments, suggesting that FVIII immune conjugates such as STALs can be useful in preventing and eliminating harmful antibody responses in humans.

The present invention accordingly provides methods and compositions for suppressing undesired immune responses and inducing systemic immune tolerance to FVIII. Some embodiments of the invention are directed to FVIII immune conjugates or compounds which contain a Factor VIII (FVIII) protein or antigenic fragment thereof that is linked to or associated with a binding moiety for a sialic acid binding Ig-like lectin (Siglec), e.g., CD22 or Siglec 10/G expressed on B cells. Some other embodiments of the invention relate to suppressing immune responses and inducing tolerance to FVIII in a subject by administering a FVIII immune conjugate containing a FVIII protein (or antigenic fragment thereof) that is conjugated to a binding moiety for a B cell Siglec (e.g., CD22 or Siglec 10/G). Some other embodiments relate to treating or preventing a bleeding disorder (e.g., hemophilia A) with FVIII deficiency in a subject by administering the noted immune conjugate to induce tolerance and co-administering an unconjugated FVIII protein or variant with coagulation activity.

The following sections provide more detailed guidance for practicing the invention.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). Further clarifications of some of these terms as they apply specifically to this invention are provided herein.

The term “agent” includes any substance, molecule, element, compound, entity, or a combination thereof. It includes, but is not limited to, e.g., protein, polypeptide, small organic molecule, polysaccharide, polynucleotide, and the like. It can be a natural product, a synthetic compound, or a chemical compound, or a combination of two or more substances. Unless otherwise specified, the terms “agent”, “substance”, and “compound” are used interchangeably herein.

The term “derivative” or “variant” is used herein to refer to a molecule that structurally resembles a reference molecule (e.g., a known Siglec ligand or a FVIII protein) but which has been modified in a targeted and controlled manner, by replacing a specific substituent of the reference molecule with an alternate substituent. Compared to the reference molecule, a derivative or variant would be expected, by one skilled in the art, to exhibit the same, similar, or improved utility. Synthesis and screening of analogs to identify variants of known compounds having improved traits (such as higher binding affinity for a target molecule) is an approach that is well known in pharmaceutical chemistry.

The term antigen broadly refers to a molecule that can be recognized by the immune system. It encompasses proteins, polypeptides, polysaccharides, small molecule haptens, nucleic acids, as well as lipid-linked antigens (polypeptide- or polysaccharide-linked lipids.

T cell-dependent or T-dependent antigens refer to antigens which require T cell assistance in eliciting antibody production by B cells. Structurally these antigens are characterized by multiple antigenic determinants. Proteins or polypeptides are typical examples of T-dependent antigens that contain antigenic determinants for both B and T cells. With a T-dependent antigen, the first signal comes from antigen cross linking of the B cell receptor (BCR) and the second signal comes from co-stimulation provided by a T cell. T-dependent antigens contain antigenic peptides that stimulate the T cell. Upon ligation of the BCR, the B cell processes the antigen, releasing antigenic peptides that are presented on B cell Class II MHC to a special subtype of T cell called a Th2 cell. The Th2 cell then secretes potent cytokines that activate the B cell. These cytokines trigger B cell proliferation, induce the B cells to produce antibodies of different classes and with increased affinity, and ultimately differentiate into antibody producing plasma cells.

T cell-independent or T-independent (TI) antigens are antigens which can directly stimulate the B cells to elicit an antibody response, do not contain proteins, and cannot induce T cell help. Typically, T-independent antigens have polymeric structures, e.g., the same antigenic determinant repeated many times. Examples of T-independent antigens include small molecule haptens, nucleic acids, carbohydrates and polysaccharides.

Bleeding disorders are a group of conditions that result when the blood cannot clot properly. In normal clotting, platelets stick together and form a plug at the site of an injured blood vessel. Proteins in the blood called clotting factors (including Factor VIII) then interact to form a fibrin clot, which holds the platelets in place and allows healing to occur at the site of the injury while preventing blood from escaping the blood vessel. Bleeding disorders suitable for treatment with the compositions and methods of the invention are preferably those which are mediated by or associated with congenital or acquired deficiencies of FVIII. Hemophilia A is perhaps the most well-known bleeding disorder. It affects mostly males.

Hemophilia is a group of hereditary genetic disorders that impair the body's ability to control blood clotting or coagulation, which is used to stop bleeding when a blood vessel is broken. Hemophilia A (clotting factor VIII deficiency) is the most common form of the disorder, present in about 1 in 5,000-10,000 male births. Hemophilia B (factor IX deficiency) occurs in around 1 in about 20,000-34,000 male births. Like most recessive sex-linked, X chromosome disorders, hemophilia is more likely to occur in males than females. This is because females have two X chromosomes while males have only one, so the defective gene is guaranteed to manifest in any male who carries it. In addition, there is the non-sex-linked hemophilia C due to coagulant factor XI deficiency, which can affect either sex. Hemophilia C is more common in Jews of Ashkenazi (east European) descent but rare in other population groups.

As used herein, immune tolerance (or simply “tolerance”) is the process by which the immune system does not attack an antigen. It occurs in three forms: central tolerance, peripheral tolerance and acquired tolerance. Tolerance can be either “natural” or “self tolerance”, where the body does not mount an immune response to self antigens, or “induced tolerance”, where tolerance to antigens can be created by manipulating the immune system. When tolerance is induced, the body cannot produce an immune response to the antigen. Mechanisms of tolerance and tolerance induction are complex and poorly understood. As is well known in the art (see, e.g., Basten et al., Curr. Opinion Immunol. 22:566-574, 2010), known variables in the generation of tolerance include the differentiation stage of the B cell when antigen is presented, the type of antigen, and the involvement of T cells and other leukocytes in production of cytokines and cofactors. Thus, suppression of B cell activation cannot be equated with immune tolerance. For example, while B cell activation can be inhibited by cross-linking CD22 to the BCR, the selective silencing of B cells does not indicate induction of tolerance. See, e.g., Nikolova et al., Autoimmunity Rev. 9:775-779, 2010; Mihaylova et al., Mol. Immunol. 47:123-130, 2009; and Courtney et al., Proc. Natl. Acad. Sci. 106:2500-2505, 2009.

The term “immune conjugate” as used herein refers to a complex in which a Siglec ligand (or binding moiety for a Siglec) is coupled to an antigen (e.g., a FVIII protein or antigenic fragment). The Siglec ligand can be coupled directly to the antigen via an appropriate linking chemistry. Alternatively, the Siglec ligand is linked indirectly to the antigen, e.g., via a third molecule such as a spacer or a lipid moiety on a liposome. The linkage between the antigen and the Siglec ligand can be either covalent or non-covalent.

A “liposomal composition” (or “liposome conjugate”) as used herein refers to a complex that contains a lipid component that forms a bilayer liposome structure. It is typically a semi-solid, ultra fine vesicle sized between about 10 and about 200 nanometers. The liposomal composition displays on or incorporates into the lipid moiety a binding moiety (e.g., a glycan ligand) that is specific for a target molecule (e.g., a Siglec) on a target cell. Typically, the binding moiety is integrated into the lipid component of the liposome complex. The liposomal composition additionally also displays a biological agent (e.g., a FVIII antigen) that is to be delivered to a target cell. The biological agent is typically also integrated into the lipid component of the liposome complex. Unless otherwise noted, the biological agent (e.g., an antigen) is not present in an aqueous solution encapsulated inside the lipid bilayer of the liposome.

Siglecs, short for sialic acid binding Ig-like lectins, are cell surface receptors and members of the immunoglobulin superfamily (IgSF) that recognize sugars. Their ability to recognize carbohydrates using an immunoglobulin domain places them in the group of I-type (Ig-type) lectins. They are transmembrane proteins that contain an N-terminal V-like immunoglobulin (IgV) domain that binds sialic acid and a variable number of C2-type Ig (IgC2) domains. The first described Siglec is sialoadhesin (Siglec-1/CD169) that is a lectin-like adhesion molecule on macrophages. Other Siglecs were later added to this family, including CD22 (Siglec-2) and Siglec-G/10 (i.e., human Siglec-10 and mouse Siglec-G), which is expressed on B cells and has an important role in regulating their adhesion and activation, CD33 (Siglec-3) and myelin-associated glycoprotein (MAG/Siglec-4). Several additional Siglecs (Siglecs 5-12) have been identified in humans that are highly similar in structure to CD33 so are collectively referred to as ‘CD33-related Siglecs’. These Siglecs are expressed on human NK cells, B cells, and/or monocytes. CD33-related Siglecs all have two conserved immunoreceptor tyrosine-based inhibitory motif (ITIM)-like motifs in their cytoplasmic tails suggesting their involvement in cellular activation. Detailed description of Siglecs is provided in the literature, e.g., Crocker et al., Nat. Rev. Immunol. 7:255-66, 2007; Crocker et al., Immunol. 103:137-45, 2001; Angata et al., Mol. Diversity 10:555-566, 2006; and Hoffman et al., Nat. Immunol. 8:695-704, 2007.

Glycan ligands of Siglecs refer to compounds which specifically recognize one or more Siglecs and which comprise homo- or heteropolymers of monosaccharide residues. In addition to glycan sequences, the Siglec glycan ligands can also contain pegylated lipid moiety connected to the glycan via a linker. Examples of various Siglec glycan ligands are reported in the literature, e.g., Paulson et al., WO 2007/056525; and Blixt et al., J. Am. Chem. Soc. 130:6680-1, 2008.

Administration “in conjunction with” one or more other therapeutic agents includes simultaneous (concurrent) and consecutive administration in any order.

The term “contacting” has its normal meaning and refers to combining two or more agents (e.g., polypeptides or small molecule compounds) or combining agents with cells. Contacting can occur in vitro, e.g., combining an agent with a cell or combining two cells in a test tube or other container. Contacting can also occur in vivo, e.g., by targeted delivery of an agent to a cell inside the body of a subject.

Sialic acid is a generic term for the N- or O-substituted derivatives of neuraminic acid, a monosaccharide with a nine-carbon backbone. It is also the name for the most common member of this group, N-acetylneuraminic acid (Neu5Ac or NANA). Sialic acids are found widely distributed in animal tissues and to a lesser extent in other species, ranging from plants and fungi to yeasts and bacteria, mostly in glycoproteins and gangliosides. The amino group generally bears either an acetyl or glycolyl group, but other modifications have been described. The hydroxyl substituents may vary considerably; acetyl, lactyl, methyl, sulfate, and phosphate groups have been found. In bacterial systems, sialic acids are biosynthesized by an aldolase enzyme. The enzyme uses a mannose derivative as a substrate, inserting three carbons from pyruvate into the resulting sialic acid structure. Sialic acid-rich glycoproteins (sialoglycoproteins) bind selectin in humans and other organisms.

The term “subject” refers to any animal classified as a mammal, e.g., human and non-human mammals. Examples of non-human animals include dogs, cats, cattle, horses, sheep, pigs, goats, rabbits, and etc. Unless otherwise noted, the terms “patient” or “subject” are used herein interchangeably. Preferably, the subject is human.

The term “treating” or “alleviating” includes the administration of compounds or agents to a subject to prevent or delay the onset of the symptoms, complications, or biochemical indicia of a disease (e.g., a bleeding disorder), alleviating the symptoms or arresting or inhibiting further development of the disease, condition, or disorder. Subjects in need of treatment include those already suffering from the disease or disorder as well as those being at risk of developing the disorder. Treatment may be prophylactic (to prevent or delay the onset of the disease, or to prevent the manifestation of clinical or subclinical symptoms thereof) or therapeutic suppression or alleviation of symptoms after the manifestation of the disease.

### III. Factor VIII Immune Conjugates Containing FVIII and Siglec Ligands

The present invention provides immune-conjugates which contain a binding moiety for a Siglec (e.g., a glycan ligand of a B cell Siglec) that is directly or indirectly linked to a FVIII protein or an antigenic fragment of FVIII. The linkage between the binding moiety and the FVIII protein can be covalent or non-covalent. Examples of non-covalent conjugations include association via hydrophobic interactions and association via electrostatic interactions. In some preferred embodiments of the invention, the binding moiety is indirectly conjugated to the FVIII protein through a liposome described herein. Thus, the FVIII-containing immune conjugate in these embodiments is a liposome nanoparticle that displays both the FVIII protein or antigenic fragment and a binding moiety that specifically recognizes a Siglec on a target cell (e.g., B lymphocytes). In some other embodiments, the binding moiety is covalently bonded to the protein. The binding moiety can be covalently conjugated to the protein via various linking chemistry well known in the art or described herein.

FVIII is a large, complex glycoprotein that primarily is produced by hepatocytes. FVIII from various species are well known and characterized in the art. For example, human FVIII (hFVIII) consists of 2351 amino acids, including signal peptide, and contains several distinct domains, as defined by homology. There are three A-domains, a unique B-domain, and two C-domains. The domain order can be listed as NH2-A1-A2-B-A3-C1-C2-COOH. FVIII circulates in plasma as two chains, separated at the B-A3 border. The chains are connected by bivalent metal ion-bindings. The A1-A2-B chain is termed the heavy chain (HC) while the A3-C1-C2 is termed the light chain (LC). FVIII circulates in association with von Willebrand Factor (VWF). VWF is a large multimeric glycoprotein that serves as a carrier for FVIII and is required for normal platelet adhesion to components of the vessel wall. See, e.g., Toole et al., Nature 312: 342-7, 1984; Truett et al., DNA 4: 333-49, 1985; and Anderson et al., Proc Natl Acad Sci USA. 83(9): 2979-2983, 1986.

The FVIII protein present in the immune conjugates of the invention can be the full length native FVIII, e.g., full length human FVIII protein. Alternatively, an antigenic fragment or variant of a full length FVIII can be used. The fragment or variant can be any part or domain of the FVIII protein that is capable of evoking an immune response, esp. activating B lymphocytes in a T cell dependent manner. In some embodiments, the FVIII protein or fragment to be used in the immune conjugates of the invention may be hFVIII derived from blood plasma and/or recombinant hFVIII. In some embodiments, the employed FVIII variant may be, e.g., B domain truncated FVIII molecules. In various embodiments, the employed FVIII protein or fragment may contain conservatively substituted amino acid residues relative to a wildtype FVIII protein (e.g., native hFVIII). In other embodiments, the employed FVIII variant has an amino acid sequence that is substantially identical to the sequence of a wildtype FVIII or antigenic fragment. Thus, relative to the wildtype FVIII, the employed FVIII variant may differ in, e.g., 1, 2, 3, 4, 5, 6, 7, 8, 9, 10, or more amino acid residues of its sequence. Alternatively, it may have a sequence that is at least 90%, 95%, 96%, 97%, 98% or 99% identical to that of the native FVIII protein or antigenic fragment.

Native FVIII proteins (e.g., hFVIII) or their antigenic fragments can be obtained either commercially or recombinantly produced. For example, recombinant hFVIII and plasma derived hFVIII may be obtained from, e.g., Pfizer (New York, N.Y.), Bayer AG (Leverkusen, Germany), BDI Pharma (Columbia, S.C.) and Reliance Life Sciences (Mumbai, India). Methods for recombinant production of FVIII proteins or antigenic fragments are well known in the art. See, e.g., Pipe S W, Thromb. Haemost. 99: 840-850, 2008; Casademunt et al., Eur. J. Haematol, 89:165-176, 2012; and Kannicht et al., Thrombo. Res. 131:78-88, 2013. Many cell lines can be used in the recombinant production of FVIII protein or antigenic fragments. Suitable host cells for producing recombinant factor VIII protein are preferably of mammalian origin in order to ensure that the molecule is glycosylated. Specific cell lines that may be used in the invention include, e.g., CHO (e.g., ATCC CCL 61), COS-1 (e.g., ATCC CRL 1650), baby hamster kidney (BHK), and HEK293 (e.g., ATCC CRL 1573; Graham et al., J. Gen. Virol. 36:59-72, 1977) cell lines.

The Siglec ligands suitable for the invention include ligands for various Siglec molecules. Some preferred embodiments of the invention employ glycan ligands directed again Siglecs that are expressed on the surface of B lymphocytes. For example, the ligands can be natural or synthetic ligands that specifically recognize CD22 (Siglec-2) and/or Siglec G/10. CD22 orthologs from a number of species are known in the art. For example, amino acid sequences for human CD22 are disclosed in the National Center for Biotechnology Information (NCBI) database (http://www.ncbi.nlm.nih.gov/) at accession number NP 001762 (gi: 4502651) and also available in WO 2007/056525. Mouse CD22 is also characterized in the art, e.g., Torres et al., J. Immunol. 149:2641-9, 1992; and Law et al., J Immunol. 155:3368-76, 1995. Other than CD22, Siglec-G/10 is another Siglec expressed on the surface of B cells. Human Siglec-10 and its mouse ortholog Siglec-G are both well known and characterized in the art. See, e.g., Munday et al., Biochem. J. 355:489-497, 2001; Whitney et al., Eur. J. Biochem. 268:6083-96, 2001; Hoffman et al., Nat. Immunol. 8:695-704, 2007; and Liu et al., Trends Immunol. 30:557-61, 2009.

Various ligands of CD22 and Siglec-G/10 are known and suitable for the practice of the present invention. See, e.g., Paulson et al., WO 2007/056525; Chen et al., Blood 115:4778-86, 2010; Blixt et al., J. Am. Chem. Soc. 130:6680-1, 2008; Kumari et al., Virol. J. 4:42, 2007; and Kimura et al., J. Biol. Chem. 282:32200-7, 2007. For example, natural ligands of human CD22 such as NeuAcα2-6Galβ1-4GlcNAc, or NeuAcα2-6Galβ1-4(6-sulfo)GlcNAc can be used for targeting a FVIII antigen to human B cells. In addition, a number of synthetic CD22 ligands with improved activities are also available, e.g., 9-N-biphenylcarboxyl-NeuAcα2-6Galβ1-4GlcNAc (6′-BPCNeuAc) and 9-N-biphenylcarboxyl-NeuAcα2-3Galβ1-4GlcNAc (3′-BPCNeuAc). More specific glycan ligands for human CD22 or Siglec-10 are described in the art, e.g., Blixt et al., J. Am. Chem. Soc. 130:6680-1, 2008; and Paulson et al., WO 2007/056525. Similarly, many glycan ligands for mouse CD22 have been reported in the literature. Examples include NeuGcα2-6Galβ1-4GlcNAc (NeuGc), 9-N-biphenylacetyl-NeuGcα2-6Galβ1-4GlcNAc (^{BPA}NeuGc), and NeuGcα2-3Galβ1-4GlcNAc. Some of these CD22 ligands are also known to be able to bind to Siglec-G/10. Other than the natural and synthetic Siglec ligands exemplified herein, one can also employ derivative or analog compounds of any of these exemplified glycan ligands in the practice of the invention.

Some FVIII immune conjugates of the invention are liposome conjugates (or liposomal compositions or compounds) for inducing systemic immune tolerance to FVIII. Typically, the FVIII liposome conjugates display on the surface of a liposome both the FVIII protein or antigenic fragment and a binding moiety that specifically recognizes a Siglec on a target cell (e.g., B cell). The binding moiety is a molecule that recognizes, binds or adheres to a target Siglec molecule located in a cell, tissue (e.g. extracellular matrix), fluid, organism, or subset thereof. The binding moiety and its target molecule represent a binding pair of molecules, which interact with each other through any of a variety of molecular forces including, e.g., ionic, covalent, hydrophobic, van der Waals, and hydrogen bonding, so that the pair have the property of binding specifically to each other. Specific binding means that the binding pair exhibit binding with each other under conditions where they do not bind to another molecule. In some preferred embodiments, the binding moiety present on the liposomal composition is a glycan ligand that specifically recognizes a Siglec (e.g., CD22 or Siglec-G/10) expressed on the surface of B cells. In addition to the binding moiety, the liposome compositions of the invention also bear or display a FVIII antigen against which immune tolerance is to be induced.

The liposome component of the liposome conjugates of the invention is typically a vesicular structure of a water soluble particle obtained by aggregating amphipathic molecules including a hydrophilic region and a hydrophobic region. While the liposome component is a closed micelle formed by any amphipathic molecules, it preferably includes lipids. For example, the liposomes of the invention exemplified herein contain phospholipids such as distearoyl phosphatidylcholine (DSPC) and polyethyleneglycol-distearoyl phosphoethanolamine (PEG-DSPE). Other phospholipids can also be used in preparing the liposomes of the invention, including dipalmitoylphosphatidylcholine (DPPC), dioleylphosphatidylcholine (DOPC) and dioleylphosphatidyl ethanolamine (DOPE), sphingoglycolipid and glyceroglycolipid. These phospholipids are used for making the liposome, alone or in combination of two or more or in combination with a lipid derivative where a non-polar substance such as cholesterol or a water soluble polymer such as polyethylene glycol has been bound to the lipid.

The FVIII liposome conjugates of the invention can be prepared in accordance with methods well known in the art. For example, incorporation of a Siglec ligand and an FVIII antigen on the surface of a liposome can be achieved by any of the routinely practiced procedures. Detailed procedures for producing a liposome nanoparticle bearing a binding moiety and a FVIII antigen are also exemplified in the Examples herein. These include liposomes bearing an incorporated glycan ligand (e.g., ^{BPC}NeuAc) and also a FVIII protein or antigenic fragment. In addition to the methods and procedures exemplified herein, various methods routinely used by the skilled artisans for preparing liposomes may also be employed in the present invention. For example, the methods described in Chen et al., Blood 115:4778-86, 2010; and Liposome Technology, vol. 1, 2^{nd} edition (by Gregory Gregoriadis (CRC Press, Boca Raton, Ann Arbor, London, Tokyo), Chapter 4, pp 67-80, Chapter 10, pp 167-184 and Chapter 17, pp 261-276 (1993)) can be used. More specifically, suitable methods include, but are not limited to, a sonication method, an ethanol injection method, a French press method, an ether injection method, a cholic acid method, a calcium fusion method, a lyophilization method and a reverse phase evaporation method. The size of the liposome of the present invention is not particularly limited, and typically is preferably between 1 to 200 nm and more preferably between 10 to 100 nm in average. The structure of the liposome is not particularly limited, and may be any liposome such as unilamella and multilamella. As a solution encapsulated inside the liposome, it is possible to use buffer and saline and others in addition to water.

Other than the above described FVIII immune conjugates in which FVIII is associated with a Siglec ligand or binding moiety via a liposome, some other embodiments of the invention relate to FVIII immune conjugates which contain a FVIII protein (or antigenic fragment) that is covalently linked to a binding moiety for a Siglec (Siglec ligand). Such immune conjugates can also be readily employed for delivering the FVIII antigen to the target B cell and accordingly inducing immune tolerance to the FVIII. Some of the immune conjugates are intended to target a FVIII antigen via a glycan ligand that recognizes a Siglec (Siglec-2 or Siglec-G/10) expressed on the surface of B cells. Suitable ligands for targeting the antigen to B cells are also described herein. Conjugating a protein or polypeptide to a small binding ligand can be performed in accordance with methods well known in the art. See, e.g., Chemistry of protein conjugation and cross-linking, Shan Wong, CRC Press (Boca Raton, Fla., 1991); and Bioconjugate techniques, 2^{nd} ed., Greg T. Hermanson, Academic Press (London, U K, 2008).

Some specific techniques described in the art may be readily employed and/or modified to achieve covalent conjugation between a FVIII antigen and a binding moiety for a Siglec. See, e.g., U.S. Pat. Nos. 4,356,170 and 5,846,951; and US Publication Nos. 2007/0282096 and 2007/0191597. Suitable linkages for the covalent conjugation include a peptide bond between a carboxyl group on one of either the FVIII antigen or the binding moiety and an amine group of the other, or an ester linkage between a carboxyl group of one and a hydroxyl group of the other. Another way of achieving covalent linkage between the FVIII antigen and the binding moiety is via a Schiff base, between a free amino group on FVIII being reacted with an aldehyde group formed at the non-reducing end of the polymer by periodate oxidation (see, e.g., Jennings and Lugowski, J. Immunol. 1981; 127:1011-8; Femandes and Gregonradis, Biochim Biophys Acta. 1997; 1341; 26-34). The generated Schiff Base can be stabilized by specific reduction with NaCNBH₃ to form a secondary amine. A further alternative approach is through the generation of terminal free amino groups in the binding moiety (e.g., a glycan ligand of a Siglec) by reductive amination with NH₄Cl after prior oxidation.

In some embodiments, enzymatic conjugation methods may be used to covalently conjugate the binding moiety to the FVIII protein. Enzymatic conjugation provides a valuable tool for accessing a restricted number of amino acid residues in a protein. For example, out of the thirteen glutamine residues of the human growth hormone, only two are substrates for the microbial transglutaminase enzyme (WO06/134148). See, e.g., Fontana et al., Adv. Drug Delivery Rev. 60:13-28, 2008; and Bonora et al. (2009), Post-translational Modification of Protein Biopharmaceuticals, Wiley, 341 and references cited therein). Similar approaches can be readily designed and adapted for determining appropriate residues in FVIII that allow for enzymatic conjugation to a binding moiety described herein.

In some other embodiments, the FVIII immune conjugates or compounds of the invention may be generated by chemical conjugation between the binding moiety and the FVIII protein. The FVIII protein or fragment may be conjugated with the binding moiety using various chemical methods. For example, chemical conjugation of relevant moieties to proteins or polypeptides may be achieved using techniques like random derivatization of some specific amino acid residues of the protein (e.g., lysine residues) by acylation or reductive alkylation. Some other immune conjugates of the invention can utilize site-selective conjugation methods. Site-selective conjugation methods are able to exploit the protein structural and biological knowledge available to choose sites. As a result, the conjugation will not significantly affect the biological activity of the conjugated protein, and at the same time obtain the desired effect on stability, pharmacokinetic parameters, immunogenicity, binding to biological partners etc. Specific site-selective conjugation methods include N-terminal specific conjugation (or at least N-terminal preferential conjugation), conjugation via the introduction of a glyoxyl group at the amino-terminus of a protein, and thiol selective conjugation to an unpaired cysteine residue.

The covalent conjugation between the FVIII antigen and the binding moiety may be carried out by direct coupling the binding moiety to the protein antigen. Nevertheless, as described above, covalent conjugation of the FVIII antigen to the Siglec ligand is more often achieved through the use of a linker moiety or linker molecule. The linker moiety can be any chemical or biological agent that facilitates formation of a desired covalent bond between the FVIII antigen and the binding moiety. Examples include short peptide recognition sequence employed in some enzymatic conjugation s and reactive chemical groups introduced in chemical conjugations. One specific example of a chemical linker is MBPH (4-[4-N-Maleimidophenyl]butyric acid hydrazide) containing a carbohydrate-selective hydrazide and a sulfhydryl-reactive maleimide group (Chamow et al., J Biol Chem 1992; 267:15916-22). Other linker moieties include bifunctional reagents which can be used for linking two amino or two hydroxyl groups. For example an amino group on the binding moiety can be coupled to amino groups of the FVIII protein with reagents like BS₃ (Bis(sulfosuccinimidyl)suberate). In addition, heterobifunctional cross linking reagents like Sulfo-EMCS (N-(e-Maleimidocaproyloxy) sulfosuccinimide ester) can be used to link amine and thiol groups.

### IV. Inducing Immune Tolerance with FVIII Immune Conjugates

The need for general methodologies to induce tolerance to protein antigens is clear in the area of biotherapeutics where anti-drug antibodies (ADA) are of considerable concern. Even after extensive efforts to minimize immunogenicity of the biological therapeutics themselves, ADAs still remain an issue in not only decreasing efficacy but, more seriously, causing anaphylaxis. For example, in patients with hemophilia, inhibitory antibodies develop in approximately 20-30% of patients shortly after initiation of FVIII therapy, thereby rendering those patients unresponsive to FVIII-products. Using immune conjugates that target a FVIII antigen to B cell Siglecs, the invention provides methods for inducing antigen-specific B-cell tolerance and thereby preventing formation of neutralizing antibodies to FVIII in a subject afflicted with a bleeding disorder with congenital or acquired deficiencies of FVIII (e.g., hemophilia A). The methods can be therapeutic in nature for ameliorating symptoms in subjects who have already manifested undesired immune response to FVIII. The method can also be prophylactic in preventing the development of undesired antibody response to FVIII, e.g., in subjects who are scheduled to receive FVIII replacement.

As exemplified herein, the methods entail administering to a subject a FVIII immune conjugate (e.g., STALs), which contains a FVIII antigen that is conjugated to a binding moiety (e.g., a glycan ligand) for B cell Siglecs (e.g., CD22). The administered immune conjugate can juxtapose the Siglec (e.g., CD22), which is an inhibitory receptor for B cell activation, with the BCR in the context of an immunological synapse and induce a tolerogenic program in B-cells. As demonstrated herein, the administered immune conjugate enables tolerization to strong T-dependent antigens such as FVIII in an antigen-specific manner. Additional evidence presented herein indicates that the induced tolerance is likely the direct result of deletion of the antigen-specific B-cells from the B-cell repertoire. The therapeutic utility of the invention at inducing antigen-specific B-cell tolerization is clearly demonstrated by the embodiments detailed in the Examples below. Specifically, the immune conjugates STALs were applied to a hemophilia mouse model since anti-FVIII antibodies are a significant problem for hemophilia A patients that receive FVIII replacement therapy. Remarkably, it was observed that tolerizing mice to rhFVIII with STALs suppressed anti-FVIII antibodies after a challenge with the immunogenic liposomes. Consistent with a lack of inhibitory antibodies in these mice, infused rhFVIII successfully prevented bleeding following tail cut.

Some embodiments of the invention are directed to inducing immune tolerance to FVIII in a subject by using the FVIII conjugates wherein the FVIII antigen is conjugated to the Siglec binding moiety via a liposome. In some other methods, FVIII immune conjugates in which the antigen is directly linked to the binding moiety via a covalent linkage are used. In various embodiments, the FVIII conjugates can be used for delivering a FVIII antigen to B cells either in vitro or in vivo. Preferably, the FVIII immune conjugate bearing both the Siglec ligand and the FVIII antigen is administered to a subject in vivo. In any of these applications, the FVIII immune conjugates disclosed herein can be used alone or administered in conjunction with other known drugs in the treatment of a bleeding disorder such as hemophilia A.

### V. Treating Bleeding Disorders with Immune Conjugates and Unconjugated FVIII

Due to the ability to induce immune tolerance specifically to FVIII, the FVIII immune conjugates of the invention also allow for treatment of bleeding disorders. The invention accordingly provides various prophylactic or therapeutic applications for treating bleeding disorders such as hemophilia A. Generally, the treatment should enable a subject to obtain a desired pharmacologic and/or physiologic effect. The effect may be prophylactic in terms of completely or partially preventing the disease or sign or symptom thereof. It can also be therapeutic in terms of a partial or complete cure for the disorder and/or adverse effect (e.g., bleeding) that is attributable to the disorders.

Typically, a subject afflicted with the bleeding disorder or at risk of developing the symptoms of the disorder is administered with a FVIII immune conjugate disclosed herein in conjunction with an unconjugated FVIII protein or variant with functional coagulation activity. The unconjugated FVIII protein to be co-administered to a subject can be a full length FVIII protein described above, e.g., full length hFVIII. The FVIII protein can be either recombinantly produced or plasma derived. In other embodiments, a FVIII variant with similar or improved coagulating function may be employed in the treatment. Thus, the unconjugated FVIII or variant should possess substantially the same proteolytic function as that of the native or wildtype FVIII, e.g., the ability to function in the coagulation cascade in a manner functionally similar or equivalent to FVIII, induce the formation of FXa via interaction with FIXa on an activated platelet, and support the formation of a blood clot. The activity can be assessed in vitro by techniques well known in the art such as, e.g. chromogenic assay, clot analysis, endogenous thrombin potential analysis, and etc. FVIII functional variants suitable for the invention should have FVIII coagulating activity being at least about 50%, at least 60%, at least 70%, at least 80%, at least 90%, and 100% or even more than 100% of that of native human FVIII.

In some embodiments, the unconjugated FVIII used in the therapeutic or prophylactic methods of the invention is a full length native hFVIII. Full length recombinant human FVIII can be obtained from several commercial sources (See, e.g., Fanchini et al., Semin. Thromb. Hemost. 36:493-7, 2010). These include first-, second- and third-generation rFVIII products. First-generation rFVIII concentrates are FVIII stabilized with human albumin. Second-generation rFVIII products contain sucrose instead of albumin in the final formulation. Finally, third-generation rFVIII products are manufactured without additional human or animal plasma proteins.

In some other embodiments, FVIII variants with functional coagulating activity are employed. For example, an unconjugated B-domain truncated/deleted FVIII can be used in conjugation with the FVIII immune conjugate of the invention for treating or preventing the development of the symptoms of hemophilia A. The exact function of the heavily glycosylated B-domain of FVIII is unknown. Nevertheless, it has been shown that this domain is dispensable for FVIII activity in the coagulation cascade. See, e.g., Sandberg et al., Semin. Hematol. 38: 4-12, 2001. This is supported by the fact that B domain deleted/truncated FVIII appears to have in vivo properties identical to those seen for full length native FVIII.

The FVIII immune conjugate and the unconjugated FVIII can be administered to a subject either sequentially or simultaneously. In some embodiments, the immune conjugate is administered first to induce tolerance before the unconjugated FVIII is administered to exert coagulation effect in the subject. In some embodiments, the immune conjugate is administered to subjects who have already been administered coagulating FVIII. In these embodiments, the immune conjugate is administered to deplete antibody-producing B cells. Typically, after induction of immune tolerance to FVIII with the immune conjugate, the subjects are again administered the unconjugated coagulating FVIII. Still in some other embodiments, the immune conjugate and the unconjugated coagulating FVIII may be administered concurrently to the subjects. For example, subjects who are genetically predisposed to developing hemophilia A but have not yet have any symptoms may receive both the immune conjugates and the unconjugated FVIII simultaneously in the prophylactic manner.

### VI. Pharmaceutical Compositions

The FVIII immune conjugates and/or the unconjugated coagulating FVIII described herein can be administered directly to subjects in need of treatment. However, these therapeutic compounds are preferably administered to the subjects in pharmaceutical compositions. Pharmaceutical compositions of the invention can be prepared and administered to a subject by any methods well known in the art of pharmacy. See, e.g., Goodman & Gilman's The Pharmacological Bases of Therapeutics, Hardman et al., eds., McGraw-Hill Professional (10^{th} ed., 2001); Remington: The Science and Practice of Pharmacy, Gennaro, ed., Lippincott Williams & Wilkins (20^{th} ed., 2003); and Pharmaceutical Dosage Forms and Drug Delivery Systems, Ansel et al. (eds.), Lippincott Williams & Wilkins (7^{th} ed., 1999).

Pharmaceutical compositions of the invention contain a therapeutically effective amount of a FVIII immune conjugate and/or the unconjugated coagulating FVIII, which are formulated with at least one pharmaceutically acceptable carrier. In addition, the pharmaceutical compositions of the invention may also be formulated to include other medically useful drugs or biological agents. The pharmaceutically acceptable carrier is any carrier known or established in the art. Exemplary pharmaceutically acceptable carriers include sterile pyrogen-free water and sterile pyrogen-free saline solution. Other forms of pharmaceutically acceptable carriers that can be utilized for the present invention include binders, disintegrants, surfactants, absorption accelerators, moisture retention agents, absorbers, lubricants, fillers, extenders, moisture imparting agents, preservatives, stabilizers, emulsifiers, solubilizing agents, salts which control osmotic pressure, diluting agents such as buffers and excipients usually used depending on the use form of the formulation. These are optionally selected and used depending on the unit dosage of the resulting formulation.

A therapeutically effective amount of the therapeutic compounds varies depending upon the disorder that a subject is afflicted with, the severity and course of the disorder, whether the treatment is for preventive or therapeutic purposes, any therapy the subject has previously undergone, the subject's clinical history and response to the therapeutic compound, and other known factors of the subject such as age, weight, etc. Thus, the therapeutically effective amount or dose must be determined empirically in each case. This empirical determination can be made by routine experimentation. A typical therapeutic dose of the FVIII immune conjugates and/or the unconjugated coagulating FVIII is about 5-100 mg per dose, e.g., 10 mg per dose. For any given condition or disease, one can prepare a suitable composition which contains a FVIII immune conjugate and/or an unconjugated coagulating FVIII in accordance with the present disclosure and knowledge well known in the art, e.g., Springhouse, Physician's Drug Handbook, Lippincott Williams & Wilkins (12^{th} edition, 2007). Depending on the specific disorder and relevant conditions of the subject to be treated, single or multiple administrations of the pharmaceutical composition of the invention can be carried out with the dose levels and pattern being selected by the treating practitioner.

Pharmaceutical compositions of the invention can be administered to a subject by any appropriate route. These include, but are not limited to, oral, intravenous, parenteral, transcutaneous, subcutaneous, intraperitoneal, intramuscular, intracranial, intraorbital, intraventricular, intracapsular, and intraspinal administration. For in vivo applications, the pharmaceutical composition of the invention can be administered to the patient by any customary administration route, e.g., orally, parenterally or by inhalation. As shown in the Example below, a liposome co-displaying a FVIII antigen and a Siglec ligand can be administered to a subject by intravenous injection. In some other embodiments, the pharmaceutical composition can be administered to a subject intravascularly. A liposome useful for intravascular administration can be a small unilamellar liposome, or may be a liposome comprising PEG-2000. When the composition is parenterally administered, the form of the drug includes injectable agents (liquid agents, suspensions) used for intravenous injection, subcutaneous injection, intraperitoneal injection, intramuscular injection and intraperitoneal injection, liquid agents, suspensions, emulsions and dripping agents.

In some other embodiments, the pharmaceutical composition may be administered orally to a subject. In these embodiments, a form of the drug includes solid formulations such as tablets, coated tablets, powdered agents, granules, capsules and pills, liquid formulations such as liquid agents (e.g., eye drops, nose drops), suspension, emulsion and syrup, inhales such as aerosol agents, atomizers and nebulizers, and liposome inclusion agents. In still some other embodiments, the pharmaceutical composition can be administered by inhalation to the respiratory tract of a patient to target the trachea and/or the lung of a subject. In these embodiments, a commercially available nebulizer may be used to deliver a therapeutic dose of the liposome compound in the form of an aerosol.

The invention also provides kits useful in therapeutic applications of the compositions and methods disclosed herein. Typically, the kits of the invention contain one or more FVIII immune conjugates and/or unconjugated FVIII described herein. The kits can further comprise a suitable set of instructions relating to the use of the compounds for inducing immune tolerance to a FVIII and/or for treating a bleeding disorder. The pharmaceutical composition of the invention can be present in the kits in any convenient and appropriate packaging. The instructions in the kits generally contain information as to dosage, dosing schedule, and route of administration for the intended therapeutic goal. The containers of kits may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable. The kits may further include a device suitable for administering the pharmaceutical composition according to a specific route of administration.

### Examples

The following examples are offered to illustrate, but not to limit the present invention.

### Example 1

### Toleragenic Liposomes with Siglec Ligands

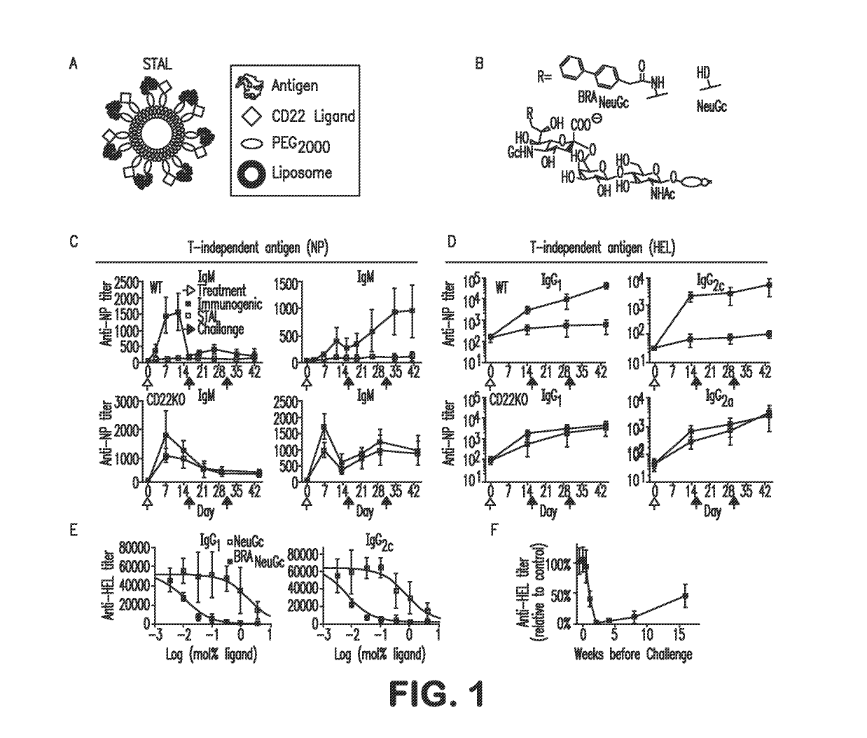

Liposomal nanoparticles were selected as a platform for enforced ligation of CD22 to the BCR because of their validated in vivo use and the robust methods that exist for covalently linking proteins and glycan ligands to lipids for incorporation into the membrane. Accordingly, Siglec-engaging tolerance-inducing antigenic liposomes (STAL) were constructed that display both CD22 ligand and antigen (FIG. 1A). The effects of STALs were compared to liposomes displaying antigen alone (immunogenic liposomes). For initial studies, we used a high affinity Siglec ligand, ^{BPA}NeuGc (^{BPA}NeuGcα2-6Galβ1-4GlcNAc; FIG. 1B), which binds to murine CD22 with 200-fold higher affinity than its natural ligand, (NeuGcα2-6Galβ1-4GlcNAc; FIG. 1B), and has only a small degree of cross-reactivity with Siglec-G.

This platform was initially validated using the T-independent antigen nitrophenol (NP) to compare with previous results using a polyacrylamide polymer. Mice injected with STALs bearing NP had a dramatically inhibited in anti-NP response (both IgM and IgG isotypes) compared to immunogenic liposomes and failed to respond to two subsequent challenges with immunogenic liposomes (FIG. 1C). In contrast, CD22KO mice treated with STALs displayed no tolerization to NP upon a subsequent challenge; thus, tolerance to NP was induced in a CD22-dependent manner.

We next formulated STALs displaying hen egg lysozyme (HEL) to investigate the potential to induce tolerance to a T-dependent antigen. Using the same experimental design, STALs induced robust tolerance of C57BL/6J mice to HEL in a CD22-dependent manner (FIG. 1D). Tolerization experiments to HEL were repeated with STALs formulated with varying amounts of either ^{BPA}NeuGc or the natural ligand, NeuGc. At the end of the 44-day experiment, involving two challenges with immunogenic liposomes on days 15 and 30, a dose-dependent effect on antibody suppression was apparent for both ligands (FIG. 1E). The two orders of magnitude difference in EC₅₀ between the two ligands is consistent with their known affinities for CD22. Maximal tolerization to HEL required two weeks to develop and diminished slowly over 4 months (FIG. 1F).

### Example 2

### STALs Induce Apoptosis of Antigen-Reactive B-Cells

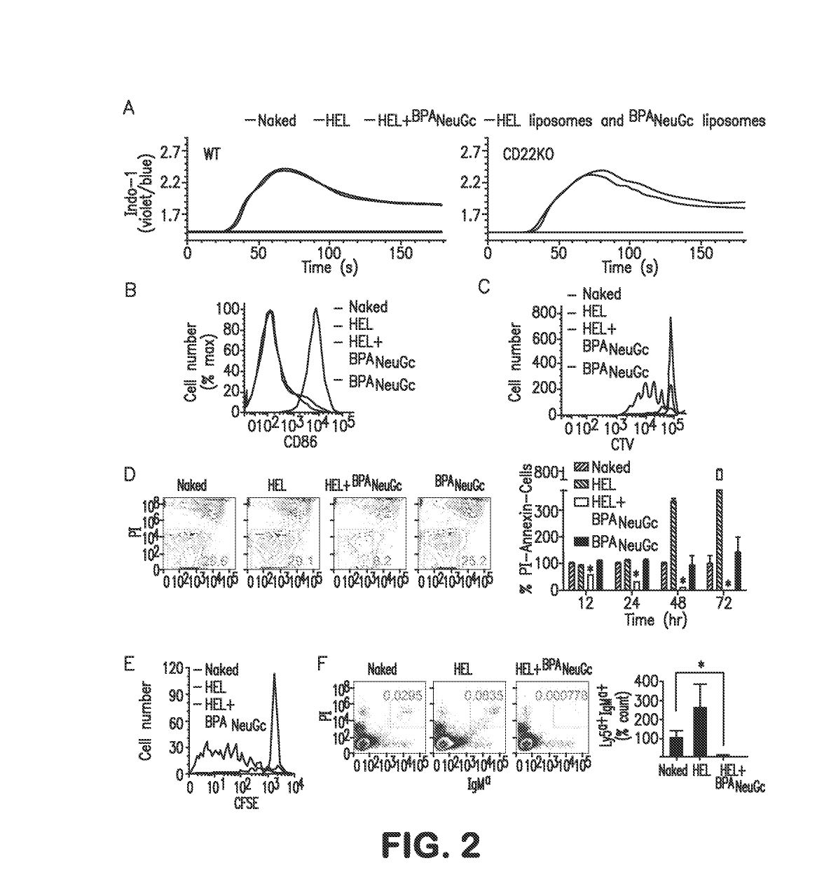

The mechanism of tolerance induction was investigated using transgenic HEL-reactive (IgM^{HEL}) B-cells from MD4 mice. STALs completely abrogated in vitro activation of IgM^{HEL} B-cells, as judged by calcium flux, CD86 upregulation, and proliferation (FIG. 2A-C). Suppressed activation was CD22-dependent as shown with IgM^{HEL} B-cells on a CD22KO background (FIG. 2A). Inhibition required presentation of both ligand and antigen on the same liposome since a mixture of liposomes displaying either CD22 ligand or antigen alone resulted in no inhibition (FIG. 2A). In proliferation assays (FIG. 2C), we noticed that cells treated with the STALs decreased in number relative to unstimulated cells. Analysis of percent live cells (AnnexinV⁻PI⁻) revealed a time-dependent decrease in this population (FIG. 2D). Culturing cells with anti-CD40, to mimic T cell help, slowed down but did not prevent cell death. It is noteworthy that liposomes displaying only CD22 ligand did not activate or affect the viability of B-cells.

Next, we examined the fate of IgM^{HEL} B-cells adoptively-transferred into host mice following immunization with liposomes. Four days after immunization, IgM^{HEL} B-cells from mice immunized with STALs had proliferated far less and were decreasing in number relative to mice immunized with naked liposomes (FIG. 2E). After 12 days, IgM^{HEL} cells (Ly5^{a+}IgM^{a+}) were depleted by greater than 95% in mice that were immunized with the STALs relative to mice that received naked liposomes (FIG. 2F). These in vivo effects were also CD22-dependent.

### Example 3

### Impact of STALs on BCR Signaling

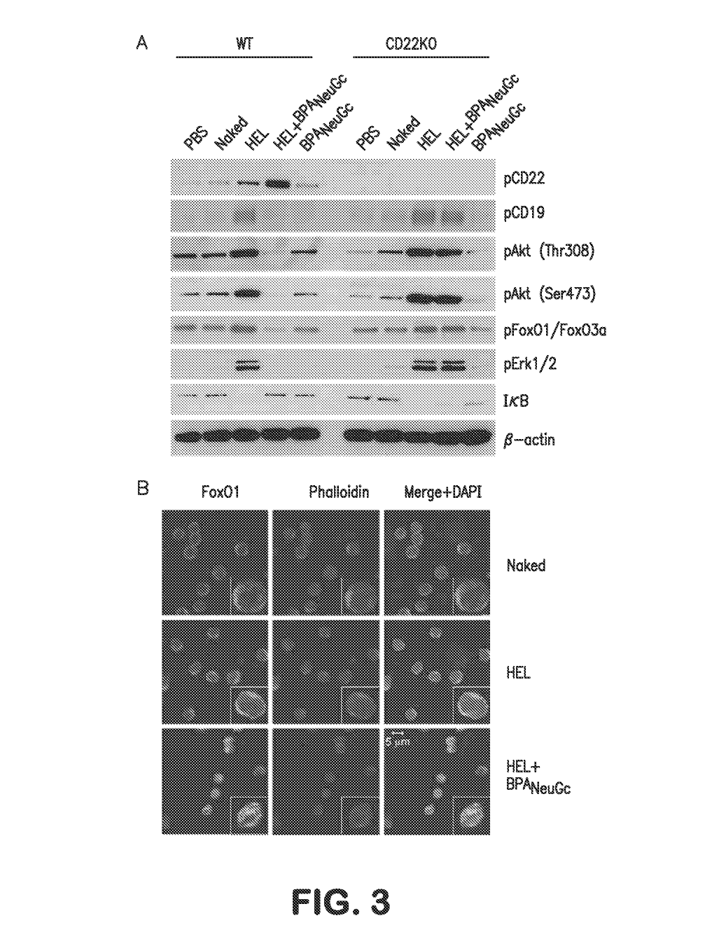

BCR signaling in IgM^{HEL} B-cells was analyzed by assessing the phosphorylation status of signaling components by Western blotting at several time points after stimulation with liposomes (FIG. 3A). STALs gave rise to strong CD22 phosphorylation on all four ITIMs analyzed, which is consistent with physical tethering of CD22 and the BCR within the immunological synapse. Conversely, phosphorylation of numerous proximal (Syk and CD19) and distal (p38, Erk, JNK, Akt, GSK3β, FoxO1, FoxO3a, BIM) BCR signaling components were strongly inhibited by the STALs compared to the liposomes displaying antigen alone at both 3 and 30-minute time points. In striking contrast, STALs and immunogenic liposomes induced equivalently strong phosphorylation of signaling components in IgM^{HEL} cells lacking CD22.

Among the affected signaling components, it is striking that STALs induced hypo-phosphorylation of components in the Akt survival pathway compared to unstimulated B-cells. Akt was hypo-phosphorylated at both the Thr308 and Ser473 sites while downstream targets of Akt, such as GSK3β and FoxO1/FoxO3a, were also hypo-phosphorylated. Given that Akt-mediated phosphorylation of the forkhead family of transcription controls their cellular location, we used confocal microscopy to analyze localization of FoxO1 and FoxO3a (FIG. 3B). FoxO1 and FoxO3a were notably absent in nuclei of resting IgM^{HEL} B-cells or cells stimulated with immunogenic liposomes, but strong nuclear staining was evident in cells treated with the STALs. As FoxO1 and FoxO3a regulate the expression of genes involved in cell cycle inhibition and apoptosis in B-cells, these results are consistent with STALs inducing a tolerogenic program involving apoptosis.

### Example 4

### Tolerance to Strong T-Dependent Antigens

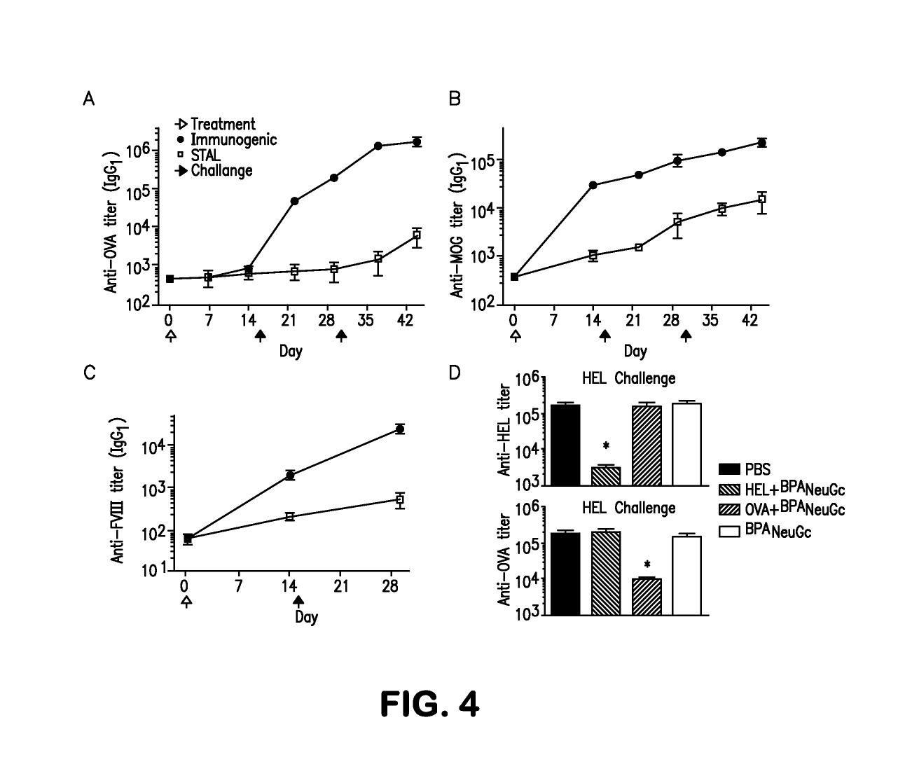

To assess the flexibility of STALs, we investigated their ability to suppress antibody production to proteins known to provide strong T cell help in C57BL/6J and/or Balb/c strains of mice. The STAL formulation was optimized to maximize CD22-mediated tolerance while minimizing T cell help by varying the amount of HEL on the liposome and titrating the amount of STALs injected during the tolerizing step. Optimized STAL formulations greatly suppressed antibody responses to HEL in Balb/c mice following a challenge with either immunogenic liposomes or soluble protein. Similarly, STALs with OVA, myelin oligodendrocyte glycoprotein (MOG), and FVIII were also tolerogenic, resulting in significantly lower antibody responses following a challenge with the corresponding antigen (FIG. 4A-4C). To assess the specificity of tolerization toward the intended antigen, we investigated the response of tolerized mice to a different antigen. Mice subjected to STALs with either HEL or OVA were found to suppress antibody production to that antigen, but have no effect on the antibody response to the other antigen (FIG. 4D). The tolerogenic impact of STALs does not appear to involve induction of suppressor cells, since adoptively-transferred splenocytes from a tolerized mouse do not suppress an antibody response to that antigen in recipient mice. Therefore, induction of antigen-specific tolerance by STALs is B-cell intrinsic.

### Example 5

### Bleeding Protection in Hemophilia Mice

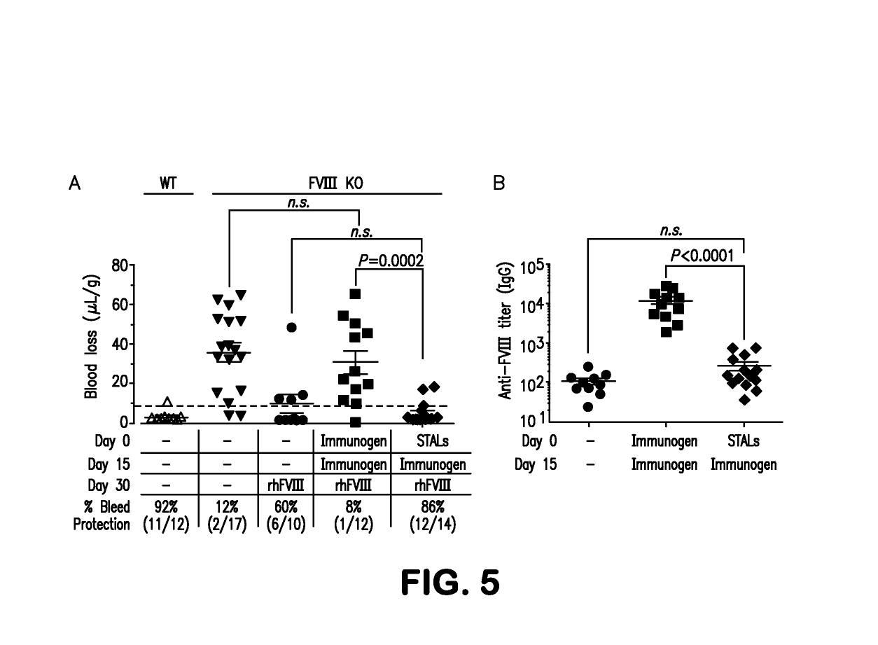

Having demonstrated that STALs suppress antibody production to human FVIII in WT mice, we investigated the impact of tolerization in FVIII-deficient mice, which serve as a model of hemophilia A. FVIII-deficient mice on a Balb/c background were used because they are highly sensitive to developing inhibitory antibodies toward FVIII, which abrogate reconstitution with FVIII to prevent bleeding. Indeed, as shown in FIG. 5A, FVIII KO mice immunized with liposomes displaying FVIII on day 0 and day 15 were unsuccessfully reconstituted with rhFVIII on day 30 since they bled to a similar extent in a tail cut experiment as FVIII KO mice that had not been reconstituted. On the other hand, mice that received STALs on day 0 followed by a challenge with immunogenic liposomes on day 15 were successfully reconstituted with FVIII and were protected from bleeding following a tail cut to a level that was statistically indistinguishable from control mice that were reconstituted with FVIII. The levels of anti-FVIII antibodies in the mice from this study correlated with the results from the bleeding assay; mice first treated with STALs prior to a challenge with immunogenic liposomes did not produce a statistically significant increase in anti-FVIII antibodies relative to control mice (FIG. 5B). In contrast, mice that received the immunogenic liposomes on day 0 and 15 had high levels of anti-FVIII antibodies. Thus, STALs are an effective means of suppressing inhibitory antibody formation against the biotherapeutic FVIII.

### Example 6

### STALs Induce Apoptosis in Human Naïve and Memory B-Cells

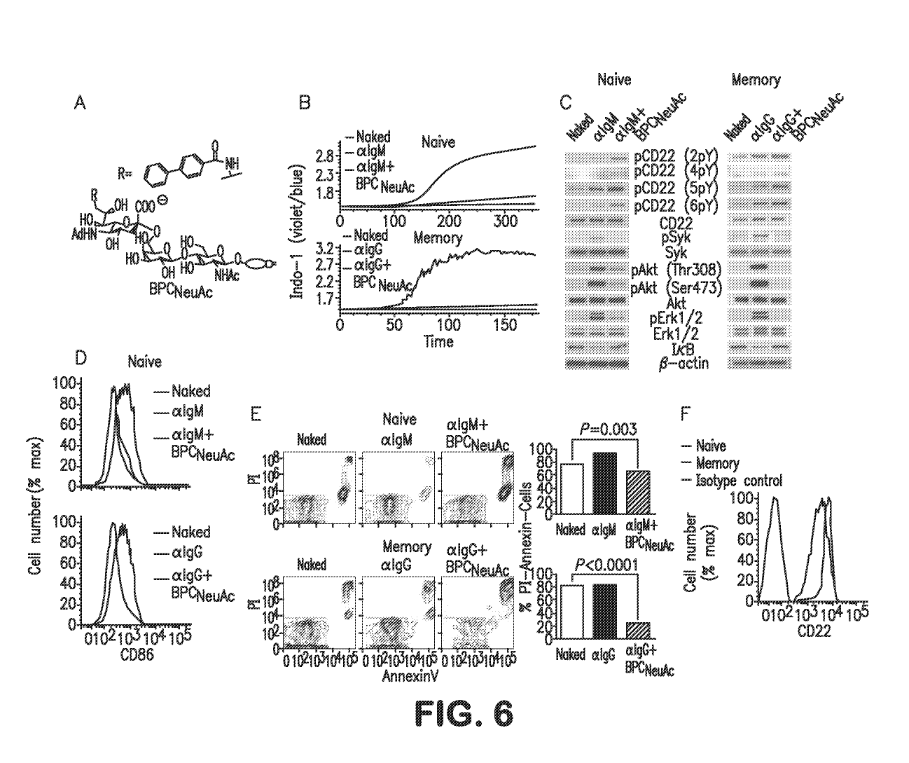

To determine if STALs similarly regulate BCR activation in human B-cells, we formulated STALs with lipid-linked anti-IgM or anti-IgG Fab fragments as surrogates of protein antigens for ligating the BCR on naïve or memory B-cells, respectively, and a high affinity human CD22 ligand termed ^{BPC}NeuAc (^{BPC}NeuGcα2-6Galβ1-4GlcNAc; FIG. 6A). Liposomes displaying anti-IgM or anti-IgG induced robust B-cell activation of naïve (CD27⁻CD38^{int}) and IgG memory (IgM⁻IgD⁻CD38⁻) B-cells isolated from peripheral blood, respectively (FIG. 6B). In contrast, liposomes displaying ^{BPC}NeuAc and the anti-Ig Fab fragments abrogated B-cell activation of both the naïve and memory cells (FIG. 6B). Similarly strong inhibition of BCR signaling was also seen in activation of BCR signaling components (FIG. 6C) and expression of CD86 (FIG. 6D). To determine if these STALs also decrease the viability of primary human B-cells, we analyzed AnnexinV and PI staining following 24 hr incubation with liposomes. The number of live cells (AnnexinV⁻PI⁻) decreased in both naïve and memory B-cells when incubated with anti-IgM or anti-IgG STALs, respectively, even in the presence of anti-CD40 (FIG. 6E). Liposomes displaying anti-IgM and ^{BPC}NeuAc or anti-IgG and ^{BPC}NeuAc had no effect on the viability of memory and naïve B cells, respectively, demonstrating that induction of apoptosis requires simultaneously engagement of the BCR and CD22. Interestingly, the STALs had a more profound effect on inhibition of B-cell activation and viability in memory B-cells despite moderately lower (2-4 fold) levels of CD22 expression in this compartment (FIG. 6F). The combined results show that the impact of STALs on BCR signaling of human B cells is similar to that observed in murine B cells, leading to apoptosis of the cells as a hallmark of tolerance induction.

### Example 7

### Some Materials and Protocols Employed in the Exemplified Studies

Mouse Strains:

CD22KO mice were obtained from L. Nitschke (University of Erlangen). WT MD4 transgenic mice were obtained from Jackson laboratories. FVIII-deficient mice (Balb/c background) were a gift of David Lillicrap (Queens University). WT C57BL/6J and Balb/c mice were obtained from the TSRI rodent breeding colony.

Proteins:

Hen egg lysozyme and ovalbumin were obtained from Sigma. MOG(1-120) was recombinantly produced in E. coli with an N-terminal polyhistidine tag for purification purposes. Briefly, residues 1-120 of rat MOG were cloned from a rat brain cDNA library (Zyagen). The PCR product was ligated into pET23a to express a protein with a C-terminal His₆-tag and purified on nickel affinity column (GE Healthcare). Recombinant human FVIII (rhFVIII) was a gift from F. Aswad at Bayer Healthcare. Anti-human IgM and anti-human IgG Fab fragments were obtained from Jackson ImmunoResearch.

Isolation of Human B-Cells:

Normal blood was obtained from TSRI's Normal Blood Donor Service. PBMCs were isolated from heperanized blood by isolating the buffy coat using ficoll-paque plus (GE healthcare). B-cells were purified by negative selection (Miltenyi). For Western blot analysis of BCR signaling components, the purified B-cells were additionally sorted for either naïve (CD27⁻CD38^{int}) or isotype-switch memory (IgM⁻IgD⁻CD38⁻) B-cells.

Immunization and Blood Collection:

Whole blood (50 μL) was collected from mice via a retro-orbital bleed to obtain the serum after centrifugation (17,000 g, 1 min). Serum was aliquoted and stored at −20° C. Liposomes were delivered via the lateral tail vein in a volume of 200 μL. For studies involving a challenge with soluble (non-liposomal) antigen, mice were injected with 200 μg of HEL dissolved in HBSS and delivered intraperitoneally.

Bleeding Assays in FVIII-Deficient Mice:

Mice were reconstituted with 200 μL of recombinant human FVIII (rhFVIII; Kogenate, Bayer Healthcare) or saline one hour prior to tail cut. rhFVIII was dosed at 50 U/Kg using a retro-orbital intravenous injection. Following one hour, mice were anesthetized and the distal portion of the tail was cut at 1.5 mm diameter and immersed in a predefined volume of saline for 20 min. The solution of saline was maintained at 37° C. Hemoglobin concentration in the solution was determined after red cell lysis with 2% acetic acid and quantified by A₄₀₅. Hemoglobin concentration against a known standard was used to calculate blood loss per gram mouse weight and expressed in μL/g, assuming a hematocrit of 46% for a normal mouse. Blood loss in WT Balb/c mice injected with 200 μL saline served as a control. Mice were considered protected if blood loss was below the mean blood loss plus three standard deviations observed in WT Balb/c mice.

Fluorescent Labeling of B-Cells:

B-cells were purified by negative selection using magnetic beads (Miltenyi). Purified IgM^{HEL} B-cells (10×10⁶ cells/ml) were fluorescently-labeled with either CFSE (6 μM) or CTV (1.5 μM) (Invitrogen) in HBSS (7 min, RT) with mixing every two minutes. Reactions were quenched by the addition of HBSS containing 3% FBS and centrifuged (270 g, 7 min) and washed a second time to remove excess labeling reagent.

In Vitro B-Cell Assays:

Purified B-cells were incubated (1 hr, RT) in media (RPMI, 10% FCS) prior to beginning the assay. Cells (0.2×10⁶) were plated in U-bottom 96-well culture plates (Falcon). Liposomes (5 μM lipid final concentration) were added and cells were incubated (37° C.) for various lengths of time. For flow cytometry analysis, cells were centrifuged (270 g, 7 min) and incubation with the appropriate antibodies in 50 μL of FACS buffer (HBSS containing 0.1% BSA and 2 mM EDTA). After staining (30 min, 4° C.), cells were washed once with 220 μL of FACS buffer and resuspended in FACS buffer containing 1 μg/mL propidium iodide prior to analyzing by flow cytometry. One exception was AnnexinV staining, which was carried out in buffer supplied by the manufacturer (Biolegend). Flow cytometry was carried out on a FACS Calibur flow cytometer (BD) and LSRII flow cytometer (BD). Labeled antibodies for flow cytometery were obtained from Biolegend and BD Biosciences.

In Vivo B-Cell Proliferation Assays:

CFSE-labeled IgM^{HEL} cells were resuspended at a concentration of 10×10⁶ cells/mL in HBSS and 200 μL (2×10⁶ cells) were injected into recipient mice via the tail vein. The following day, liposomes were injected via the tail vein. Four days later, the spleens of the recipient mice were harvested to analyze the CFSE staining of Ly5^{a+}IgM^{a+} B-cells.

Calcium Flux:

Purified B-cells were resuspended at 15×10⁶ cells/mL in RPMI media containing 1% FCS, 10 mM HEPES, 1 mM MgCl₂, 1 mM EGTA, and 1 μM Indo-1 (Invitrogen). Cells were incubated in a 37° C. water incubator for 30 minutes. Following incubation (37° C., 30 min), a five-fold volume of the same buffer (without Indo-1) was added and the cells were centrifuged (270 g, 7 min). For experiments involving human B-cells, cells were stained with the appropriate antibodies for 20 min on ice in HBSS containing 3% FCS. To analyze human naive B-cells, the cells were stained with anti-CD27 and anti-CD38. To analyze human memory B-cells, cells were stained with anti-CD38, anti-IgM, and anti-IgD. Cells were washed and resuspended at a concentration of 2×10⁶ cells/mL in HBSS containing 1% FCS, 1 mM MgCl₂, and 1 mM CaCl₂. Cells were stored on ice and an aliquot (0.5 mL; 1×10⁶ cells) was warmed (37° C., 5 min) prior to initiating calcium flux measurements. Cells were stimulated with liposomes (ranging from 5-50 μM) and Indo-1 fluorescence (violet vs. blue) was monitored by flow cytometry (500-1000 events/sec) for 3-6 minutes at 37° C. Stimulation always took place 10 sec. after starting acquisition so that background could be established. Data was analyzed in FlowJo using the kinetics functions.

ELISAs:

Maxisorp plates were coated (0/N, 4° C.) with the relevant protein (50 μL/well, 10 μg/mL) in PBS. NP₄₋₇-BSA in PBS (Biosearch Technologies) was used to look for anti-NP antibodies. The following day, plates were washed twice in TBS-T (0.1% Tween 20) and blocked (1 hr, RT) with TBS-T containing 1% BSA. Serum was initially diluted between 20-10,000-fold and diluted in 2-3 fold serial dilutions eight times on the ELISA plate. Plates were incubated (1 hr, 37° C.) with serum (50 μL/well), washed four times, and incubated (1 hr, 37° C.) with the appropriate HRP-conjugated secondary antibodies (1:2000, Santa Cruz Biotechnologies). Following five washes, plates were developed (RT, 15 min) in 75 μL/well of TMB substrate (Thermo Fisher) and quenched with 75 μL/well of 2N H₂SO₄. Absorbance was measured at 450 nm and the endpoint titer was calculated as the dilution of serum that produced an absorbance 2-fold above background.

Western Blotting:

Purified B-cells (30×10⁶/condition) were incubated (37° C., 1 hr) in media (RPMI, 3% FCS) prior to stimulating the cells. Liposomes (5 μM lipid final concentration) were added to cells and after a 3 or 30 minute incubation (37° C.), cells were centrifuged (13,000 g, 8 sec), washed with cold PBS, centrifuged, and lysed (4° C., 30 min) in 280 μL of lysis buffer (20 mM Tris, 150 NaCl, 1 mM EDTA, 1% Triton-X 100, 10 mM NaF, 2 mM Sodium orthovanadate, protease inhibitor cocktail (Roche), pH 7.5). Cell debris was removed by centrifugation (13,000 g, 10 mM, 4° C.). SDS-PAGE loading buffer was added and samples denatured (75° C., 15 min). Samples were run on 4-12% gradient SDS-PAGE gels (Invitrogen) and transferred to nitrocellulose. Membranes were blocked (RT, 1 hr) in 5% nonfat milk powder dissolved in TBS-T and probed with primary antibody (0/N, 4° C.) in TBS-T containing 1% BSA. Primary antibodies were obtained from Cellular Signaling Technologies and used at dilution of 1:1000. Phosphospecific CD22 antibodies were a gift from M. Fujimoto (University of Tokyo)(50). Next day, membranes were washed (4×5 min), blocked (30 min, RT) and probed (1 hr, RT) with secondary HRP-conjugated antibodies (1:10,000 dilution; Santa Cruz Biotechnologies). Following four washes, blots were incubated (2 min, RT) with developing solution (GE Healthcare) and exposed to film.

Microscopy:

Purified IgM^{HEL} B-cells were stimulated in the same manner as the Western blot analysis for 2 hr. Following stimulation, cells were pelleted (0.5 g, 3 min), washed with cold PBS, and again gently centrifuged. The pellet was resuspended in 1 mL of cold 4% paraformaldehyde (PFA) and rotated (4° C., 10 min). Cells were gently centrifuged and the pellet resuspended in 2004 of PBS. Resuspended cells (50 μL, 3×10⁶ cells) were dispersed onto poly-lysine slides (Fisher). After drying, the slides were washed three times with PBS, permeabilized with 5% Triton-X 100 (5 min, RT), followed by blocking with 5% normal goat serum (NGS) (30 min, RT). Slides were probed with anti-FoxO1 or anti-FoxO3a (Cellular Signaling Technologies) at a concentration of 1:80 in solution of 1% NGS containing 0.01% TX-100 (O/N, 4° C.). Next day, slides were wash three times with PBS and probed with Alexa488-conjugated goat anti-rabbit (1:1000; Invitrogen) and Alexa555-phalloidin (1:40; Invitrogen) in 1% NGS. Following three washes with PBS, slides were incubated with a solution of DAPI and mounted in Prolong anti-fade medium (Invitrogen). Imaging of the cells was carried out on a Zeiss confocal microscope.

Protein-Lipid Conjugation:

Proteins were conjugated to pegylated distearoylphosethanolamine (PEG-DSPE) using maleimide chemistry. A thiol group was introduced using the heterobifunctional crosslinker N-succinimidyl 3-(2-pyridyldithio)-propionate (SPDP; Pierce). Approximately 2.5 molar equivalents of SPDP (in DMSO) were added to a protein solution (in PBS). The reaction was gently rocked (RT, 1 hr). The protein was desalted on a sephadex G-50 column and treated with 25 mM DTT (10 min, RT). The amount of released thiol 2-pyridyl group was quantified by absorbance at 343 nm to calculate the extent of protein modification. Following desalting, the thiol-derivatized protein (in the range of 5-50 μM) was immediately reacted with Maleimide-PEG₂₀₀₀-DSPE (200 μM; NOF America) under nitrogen (RT, O/N). Lipid-modified proteins were purified from unmodified protein on a sephadex G-100 column and stored at 4° C. SDS-PAGE was used to verify the proteins were modified by lipid by an increase in their apparent MW on the gel. Using these reaction conditions, proteins were modified with between one to three lipids.

Sugar-Lipid Conjugation:

The high affinity murine CD22 ligand (^{BPA}NeuGc) and human CD22 ligand (^{BPC}NeuAc) were attached to PEG-DSPE by coupling 9-N-biphenylacetyl-NeuGcα2-6Galβ1-4GlcNAc-β-ethylamine or 9-N-biphenylcarboxyl-NeuAcα2-6Galβ1-4GlcNAc-β-ethylamine to NHS-PEG₂₀₀₀-DSPE (NOF), respectively, as described previously. NP-PEG₂₀₀₀-DSPE was synthesized under similar conditions through 4-Hydroxy-3-nitrophenylacetyl-O-succinimide with amine-PEG₂₀₀₀-DSPE (NOF).

Liposomes:

All liposomes were composed of a 60:35:5 molar ratio of distearoyl phosphatidylcholine (DSPC; Avanti Polar Lipids), cholesterol (Sigma), and pegylated lipids. The total mol % of pegylated lipids was always kept at 5%; made up of the appropriate combination of polyethyleneglycol(PEG₂₀₀₀)-distearoyl phosphoethanolamine (PEG-DSPE; Avanti Polar Lipids), ^{BPA}NeuGc-PEG₂₀₀₀-DSPE, ^{BPC}NeuAc-PEG₂₀₀₀-DSPE, NP-PEG₂₀₀₀-DSPE or Protein-PEG₂₀₀₀-DSPE. To assemble the liposomes, DSPC and cholesterol (dissolved in chloroform) were evaporated under nitrogen. ^{BPA}NeuGc-PEG₂₀₀₀-DSPE, ^{BPC}NeuAc-PEG₂₀₀₀-DSPE, NP-PEG₂₀₀₀-DSPE, from DMSO stocks, was added to the dried lipid and this mixture was lyophilized. The dried lipids were hydrated in PBS (1-10 mM lipid) and sonicated vigorously for a minimum of 5×30 s. Protein-PEG₂₀₀₀-DSPE was added at the time of hydration. The mol % of the protein on the liposome was varied during our studies from 0.0033-0.33%. Liposomes were passed a minimum of 20 times through 800 nm, 200 nm, and 100 nm filters using a hand-held mini-extrusion device (Avanti Polar Lipids). Extrusion was carried at 40-45° C. The diameter of the liposomes were measured on a zetasizer (Malvern) and were consistently in the range of 100-130±30 nm. For studies with NP as the antigen, liposomes contained 0.5 mol % NP (concentration based on lipid content). Mice received 200 μl of 2.5 mM liposomes. For studies with HEL as the antigen in C57BL/6J mice, liposomes contained 0.1 mol % and mice received 200 μl of 1 mM liposomes. For studies with HEL as the antigen in Balb/C mice, the mol % and absolute amount of liposomes used during the immunization were optimized. Optimized conditions, which were also used for OVA, MOG, and FVIII, contained 0.01 mol % and mice received 200 μl of 10 μM liposomes. For in vitro experiments with IgM^{HEL} B cells and human primary B cells, liposomes contained 0.1 mol % HEL and anti-Ig, respectively, and cells were incubated with 10 μM liposomes. All STALs contained 1 mol % CD22 ligand, except in FIG. 1E where the ligand mol ratio was titrated.

Statistical Analyses:

Statistical significance was determined using an unpaired two-tailed Student's t-test. A P value less than 0.05 was considered significant.

The invention thus has been disclosed broadly and illustrated in reference to representative embodiments described above. It is understood that various modifications can be made to the present invention without departing from the spirit and scope thereof. It is further noted that all publications, patents and patent applications cited herein are hereby expressly incorporated by reference in their entirety and for all purposes as if each is individually so denoted. Definitions that are contained in text incorporated by reference are excluded to the extent that they contradict definitions in this disclosure.

1. A compound comprising a Factor VIII (FVIII) protein or antigenic fragment thereof that is conjugated to a binding moiety for a sialic acid binding Ig-like lectin (Siglec).

2. The compound of claim 1, wherein the FVIII or antigenic fragment thereof is conjugated to the binding moiety via a liposome.

3. The compound of claim 1, wherein the FVIII or antigenic fragment thereof is covalently conjugated to the binding moiety.

4. The compound of claim 1, wherein the FVIII is human FVIII.

5. The compound of claim 1, wherein the Siglec is a Siglec expressed on B lymphocytes.

6. The compound of claim 1, wherein the Siglec is CD22 or Siglec-G/10.

7. The compound of claim 1, wherein the binding moiety comprises a glycan ligand for the Siglec.

8. The compound of claim 7, wherein the glycan ligand is 9-N-biphenylcarboxyl-NeuAcα2-6Galβ1-4GlcNAc (6′-BPCNeuAc), NeuAcα2-6Galβ1-4GlcNAc, or NeuAcα2-6Galβ1-4(6-sulfo)GlcNAc.

9. A method for inducing immune tolerance to Factor VIII (FVIII) in a subject, comprising administering to the subject a therapeutically effective amount of a compound comprising a Factor VIII protein or antigenic fragment thereof that is conjugated to a binding moiety for a sialic acid binding Ig-like lectin (Siglec) expressed on B lymphocytes, thereby inducing immune tolerance to FVIII in the subject.

10. The method of claim 9, wherein the FVIII or antigenic fragment thereof is conjugated to the binding moiety via a liposome.

11. The method of claim 9, wherein the FVIII or antigenic fragment thereof is covalently conjugated to the binding moiety via a linker.

12. The method of claim 9, wherein the Siglec is CD22 or Siglec-G/10.

13. The method of claim 9, wherein the binding moiety comprises a glycan ligand for the Siglec.

14. The method of claim 13, wherein the glycan ligand is 9-N-biphenylcarboxyl-NeuAcα2-6Galβ1-4GlcNAc (6′-BPCNeuAc), NeuAcα2-6Galβ1-4GlcNAc, or NeuAcα2-6Galβ1-4(6-sulfo)GlcNAc.

15. (canceled)

16. (canceled)

17. The method of claim 9, wherein the subject is afflicted with a bleeding disorder.

18. The method of claim 17, wherein the subject is afflicted with hemophilia A.

19. The method of claim 9, wherein the compound is administered to the subject in a pharmaceutical composition.

20. A method for treating hemophilia A, comprising administering to a subject in need of treatment (1) a therapeutically effective amount of a conjugate compound that comprises a Factor VIII (FVIII) protein that is conjugated to a glycan ligand for a B lymphocyte sialic acid binding Ig-like lectin (Siglec), and (2) an unconjugated FVIII protein or variant with coagulation activity, thereby treating hemophilia A in the subject.

21. The method of claim 20, wherein the conjugate compound is administered to the subject prior to administration of the unconjugated FVIII protein or variant.

22. The method of claim 20, wherein the FVIII protein in the conjugate compound is conjugated to the glycan ligand via a liposome.

23. The method of claim 20, wherein the FVIII protein in the conjugate compound is covalently conjugated to the glycan ligand via a linker.

24-29. (canceled)
